(19) United States
(12) Patent Application Publication
(10) Pub. No.: US 2021/0023232 A1
(43) Pub. Date: Jan. 28, 2021

(54) COMBINATION OF POLYETHYLENE GLYCOL AND RAPAMYCIN AND USE THEREOF

(71) Applicant: JENKEM TECHNOLOGY CO., LTD. (TIANJIN), Tianjin (JP)

(72) Inventors: Jinliang WANG, Beijing (CN); Zewang FENG, Beijing (CN); Leimin WANG, Beijing (CN); Xuan ZHAO, Beijing (CN)

(73) Assignee: JENKEM TECHNOLOGY CO., LTD. (TIANJIN), Tianjin (CN)

(21) Appl. No.: 17/042,887

(22) PCT Filed: Mar. 28, 2019
(86) PCT No.: PCT/CN2019/080194

§ 371 Date: Sep. 28, 2020

(30) Foreign Application Priority Data
Mar. 29, 2018 (CN) 201810273306.2

(51) Int. Cl. A61K 47/60; A61K 31/436

(52) U.S. Cl. CPC A61K 47/60; A61K 45/06; A61K 31/436

(57) ABSTRACT

The present invention discloses a conjugate of PEG (polyethylene glycol) and rapamycin and use thereof, in particular use in preparation of a medicament for reducing immune response, wherein conjugate of PEG and rapamycin can remarkably lower the generation rate of an antibody directed to foreign immunogen, and reduce excessive immune responses caused by use thereof. The conjugate of PEG and rapamycin has beneficial effects of ensuring and even improving the treatment effect of a therapeutic agent, improving the own immunity of a subject, reducing and even eliminating graft rejection, and is advantageous in a relatively simple preparation process thereof, low cost, easy industrial production, and a high application value.

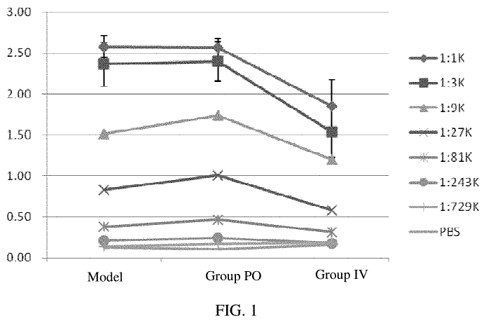

### BACKGROUND OF THE INVENTION

### 1. Technical Field

The present invention relates to the technical field of medicaments, specifically to a conjugate of PEG (polyethylene glycol) and rapamycin and use thereof, in particular use in preparation of a medicament for reducing immune response, and more specifically use of the conjugate of PEG and rapamycin in preparation of a medicament for reducing excessive immune responses caused by foreign immunogen such as a therapeutic agent and an implant.

### 2. Description of Related Art

Immunogenicity refers to a capability of stimulating body to generate a specific antibody or a sensitized lymphocyte, which means the feature that an antigen can simulate a specific immune cell to activate, reproduce, differentiate and finally generate the immune antibody and the sensitized lymphocyte. Immune response refers to a physiological process generated in response to the stimulation of the immunogen by the immune system of the body for the purpose of eliminating the antigen.

Some therapeutic agents also have immunogenicity because of respective chemical properties, for example, some macromolecular proteins and polysaccharides, micro-molecule polypeptides, nucleic acid and the like. Implants such as cells, tissues, organs and artificial materials can also induce immune response. Under such circumstance, cells in the immune system identify the therapeutic agent and implant and generate attack, damage and clearance chemical reactions, thereby the making the treatment on the patient difficult, bringing more pains to patients, even making the physical conditions of the patients worse and endangering life.

Rapamycin (also referred to as Sirolimus) is a kind of macrolides antibiotics found in soil at Easter Island of Chile in 1975. Rapamycin is generated by Streptomyces hygroscopicus. Rapamycin developed by an American company Home Products by was put into clinical use as a new medicament for suppressing transplantation rejection in 1989 and then approved to come into market by American PDA in September 1999. Rapamycin clinically shows a great prospect, but still has defects such as low biological availability and low water insolubility. In recent years, some researchers tried to reduce the foregoing excessive immune response with conjugates of rapamycin and a nano-carrier. For example, the patent application CN201480031937.3 discloses an administration composition of a therapeutic macromolecule which does not bind to a synthesized nano-carrier and an immunosuppressor which links to the synthesized nano-carrier to reduce unexpected humoral immune response.

However, the conjugate of rapamycin and the nano-carrier is complicated in preparation process, high in cost, wide in particle size range of the nano-carrier, poor in product batch stability, undesirable in treatment effect, and not good for mass industrial production and actual application.

### BRIEF SUMMARY OF THE INVENTION

To overcome defects in prior art, the present invention provides use of a conjugate of PEG (polyethylene glycol) and rapamycin in preparation of a medicament for reducing immune response. The conjugate of PEG and rapamycin has the structure shown in Formula I below:

PEG-X-D (I)

wherein,

PEG is a polyethylene glycol residue,

D is a residue of rapamycin or a derivative thereof, and

X is a linking group between PEG and D.

Preferably, in the conjugate, X is selected from one or a combination of more of —(CR₁R₂)ₐ—, —(CH₂)ₐNH—, —(CH₂)ₐNHCO—, —(CH₂)ₐCONH—, —(CH₂)ₐCO—, —(CH₂)ₐCOO—, —(CH₂)ₐO—, cycloalkyl, aryl, heterocyclic, -A- and —Op-,

wherein, a is an integer of 0-10,

each of R₁ and R₂ is indecently selected from one or a combination of more than two of —H, C₁₋₆ alkyl, —OR′, —NHR′, —N(R′)₂, —CN, —F, —Cl, —Br, —I, —COR′, —COOR′, —OCOR′, —CONHR′ and —CON(R′)₂,

R′ is selected from —H, C₁₋₆alkyl, —F, —Cl, —Br and —I,

-A- is an amino acid residue, and

—Op- is an oligopeptide residue (for example, a peptide with 2-10 (specifically for example, 2, 3, 4, 5, 6, 7, 8, 9 or 10) amino acid residues).

In one embodiment of the present invention, X has the following structure: —X₁—X₂—X₃—, wherein each of X₁ and X₃ is independently selected from one or a combination of more of —(CR₁R₂)ₐ—, —(CH₂)ₐNH—, —(CH₂)ₐNHCO—, —(CH₂)ₐCONH—, —(CH₂)ₐCO—, —(CH₂)ₐCOO— and —(CH₂)ₐO—, and X₂ is selected from cycloalkyl, aryl, heterocyclic, -A- and —Op-.

Preferably, the amino acid residue -A- is selected from one of the following structures:

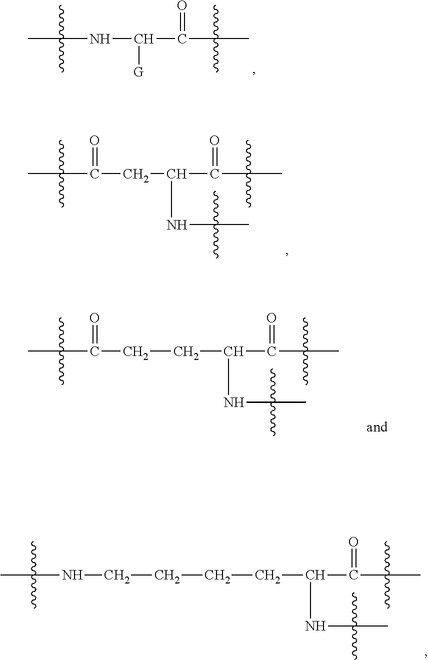

wherein, G is selected from —H, —CH₃,

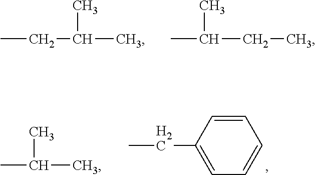

—CH₂—S—CH₃,

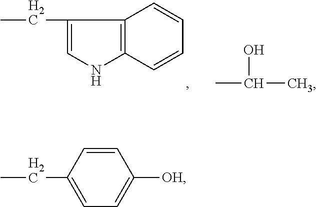

—CH₂OH, —CH₂SH, —CH₂CONH₂, —CH₂CH₂CONH₂,

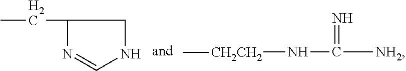

preferably selected from —H, —CH₃,

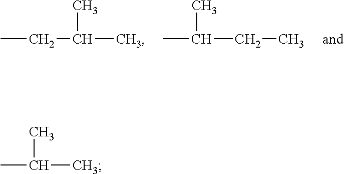

and more preferably, G is —H or —CH₃.

Preferably, the oligopeptide residue —Op- has 2-10 amino acid residues; the amino acid residues are the same or different, selected from the defined solution for the foregoing amino acid residue -A- of the present invention; the oligopeptide residue —Op- can be a linear oligopeptide residue, for example dipeptide

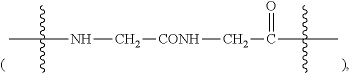

tripeptide

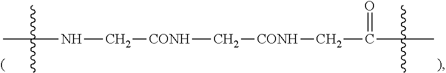

and the like of glycine, and under such circumstance, the oligopeptide residue —Op- has two linkable points; the oligopeptide residue —Op- can further be a branched oligopeptide residue for example

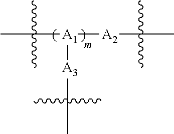

(m is 1, 2, 3 or 4), wherein A₁ is selected from a residue of aspartic acid, glutamic acid and lysine, each of A₂ and A₃ is independently same or different, selected from the defined solution for the foregoing amino acid residue -A- of the present invention, specifically for example, the multi-armed oligopeptide as described in the Chinese patent Application CN201410715522.X, in particular for example,

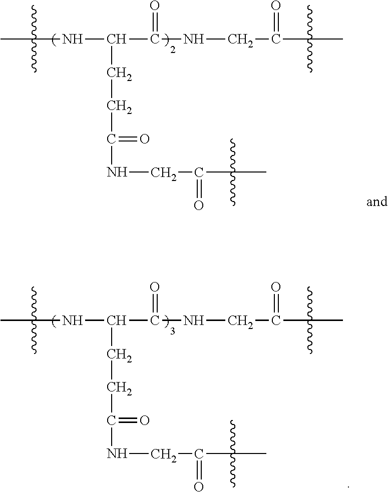

Each of R₁ and R₂ is indecently selected from one or a combination of more than two of —H, C₁₋₆ alkyl, —OR′, —NHR′, —N(R′)₂, —CN, —F, —Cl, —Br, —I, —COR′, —COOR′, —CONHR′ and —CON(R′)₂; R′ is selected from —H, C₁₋₆ alkyl, —F, —Cl, —Br and —I; and

a is an integer of 0-10,

Preferably, R′ is selected from —H and C₁₋₃ alkyl (for example, methyl, ethyl, n-propyl or isopropyl).

Preferably, each of R₁ and R₂ is independently selected from one or a combination of more of H, C₁₋₃ alkyl (for example, methyl, ethyl, n-propyl or isopropyl), —OH, C₁₋₃ alkoxy (i.e. —OR, R′ is C₁₋₃ alkyl), —NH₂, —F, —Cl, —Br and —I, more preferably selected from H, —CH₃, —OH, —OCH₃ and —OCH₂CH₃.

Preferably, a is an integer of 0-5, for example 0, 1, 2, 3, 4 or 5.

Preferably, each of X₁ and X₃ is independently one or a combination of more of single bond, —CH₂—, —CH₂CH₂—, —CH₂CH₂CH₂—, —CH₂CO—, —CH₂CH₂CO—, —CH₂NH—, —CH₂NHCH₂—, —CH₂CONH—, —CH₂CONHCH₂—, —CH₂NHCO—, —CH₂NHCOCH₂—, —CH₂COO—, —CH₂COOCH₂—, —CH₂O— and —CH₂OCH₂.

In one embodiment of the present invention, X₁ is —CH₂CO—.

In another embodiment of the present invention, X₁ is —CH₂OCH₂—.

In another embodiment of the present invention, X₁ is —CH₂CONHCH₂—.

In one embodiment of the present invention, X₃ is a single bond.

In one embodiment of the present invention, X₃ is —CH₂CO—.

In one embodiment of the present invention, —Op- has the following structure:

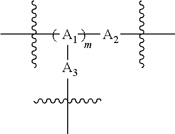

wherein, m is 1, 2 3 or 4,

A₁ is selected from a residue of aspartic acid, glutamic acid and lysine,

A₂ and A₃ are the same or different, each independently selected from the following structure:

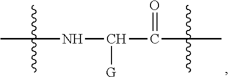

wherein G has the foregoing definition of the present invention.

In one embodiment of the present invention, A₁ is a glutamic acid residue

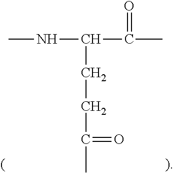

In one embodiment of the present invention, A₂ and A₃ are the same and are both a glycine residue

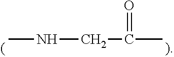

In a specific embodiment of the present invention, —Op- has the following structure:

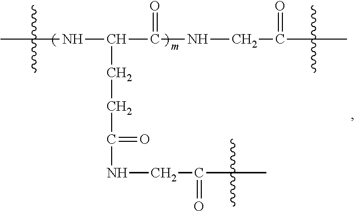

wherein preferably, m is 2 or 3. In the conjugate shown by Formula I of the present invention, the linking group X can include only two linking sites which respectively link PEG and D; X can further include more than three linking sites, which respectively link one PEG residue and more than two residues of rapamycin and derivatives thereof to fulfill the aim of increasing the amount of loaded drug, or which respectively link one residue of rapamycin and derivatives thereof and more than two PEG residues to fulfill the aim of improving the water solubility or stereospecific blockade of the conjugate.

In another embodiment of the present invention, X is —X₁—Op-X₃—, and —Op- has the foregoing definition of the present invention.

In one embodiment of the present invention, X is

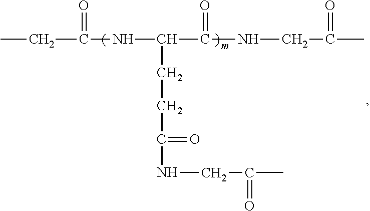

wherein preferably m is 2 or 3.

Preferably, in the conjugate, PEG is a residue of linear, Y type branched or multi-arm PEG, for example including methoxy-polyethylene glycol (mPEG), linear PEG with two terminal ends, Y-type branched PEG, four-arm PEG, six-arm PEG or eight-arm PEG.

Preferably, the molecular weight of the PEG can be 1-100 KDa, for example 1-10 KDa (specifically, 1, 2, 3, 4, 5, 6, 7, 8, 9, or 10 KDa), 10-15 KDa (specifically, 10, 15, 20, 25, 30, 35, 40, 45 or 50 KDa) or 50-100 KDa (specifically, 50, 55, 60, 65, 70, 75, 80, 85, 90, 95 or 100 KDa), and the like, further preferably 10-50 KDa.

In a specific embodiment of the present invention, PEG is a linear PEG residue, which has the structure shown in Formula II or III below:

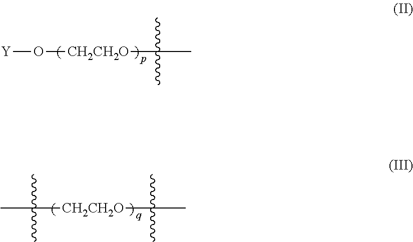

wherein, each of p and q is independently an integer of 1-2280, and preferably 220-1140,

Y is a terminal group, with a -L-T structure,

wherein, L is a linking group between oxygen (O) and a capping terminal group T, selected from one or a combination of more of (CH₂)_{b}—, —(CR₃R₄)_{b}—, —(CH₂)_{b}NH—, —(CH₂)_{b}NHCO—, —(CH₂)_{b}CONH—, —(CH₂)_{b}CO—, —(CH₂)_{b}COO— and —(CH₂)_{b}O—, wherein b is an integer of 0-10,

each of R₃ and R₄ is indecently selected from one or a combination of more of —H, C1-6 alkyl, —OR′, —NHR′, —N(R′)₂, —CN, —F, —Cl, —Br, —I, —COR′, —COOR′, —CONHR′ and —CON(R′)₂,

R′ is selected from —H, C₁₋₆alkyl, —F, —Cl, —Br and —I, and,

T is the capping terminal group, selected from a residue pf H, C1-6 alkyl, C3-6 cycloalkyl, C4-10 cycloalkylalkyl, C6-10 aryl, C7-14 aralky, monosaccharide and oligosaccharide.

Preferably, L is selected from one or a combination of more of single bond, —CH₂—, —CH₂CH₂—, —CH₂CH₂CH₂—, —CONH—, —NH—, —CO—, —CONHCH₂—, —CH₂NH—, —CH₂CONH— and —COCH₂—.

Preferably, T is selected from methyl, ethyl, isopropyl, cyclopropyl, cyclopropylmethyl, cyclobutyl, cyclohexyl, benzyl,

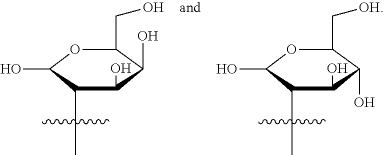

Preferably, Y is selected from methyl, ethyl, isopropyl, cyclopropyl, cyclopropylmethyl, cyclobutyl, cyclohexyl, benzyl,

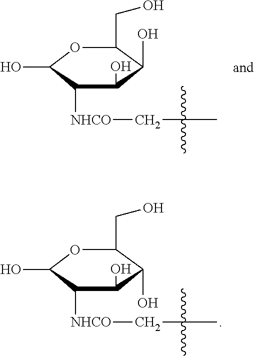

In a preferred embodiment of the present invention, Y is methyl.

In another preferred embodiment of the present invention, Y is

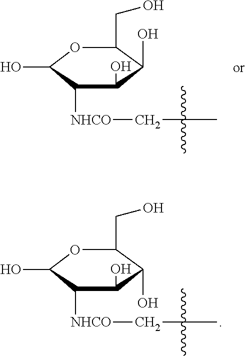

In a specific embodiment of the present invention, PEG is a Y-type branched PEG residue, which has the structure shown in Formula IV or V below:

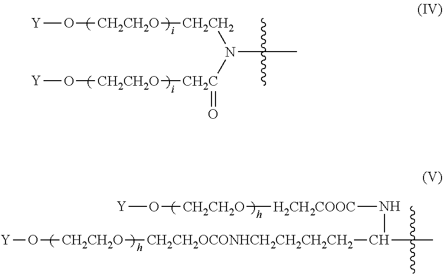

wherein, each of i and h is independently an integer of 1-1140, and preferably 110-570,

Y has the structure defined by foregoing Formula II of the present invention, preferably selected from methyl, ethyl, isopropyl, cyclopropyl, cyclopropylmethyl, cyclobutyl, cyclohexyl, benzyl,

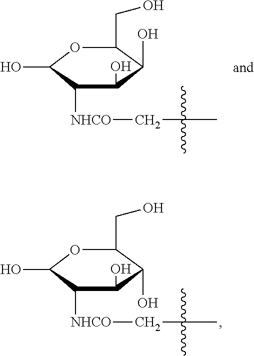

more preferably selected methyl,

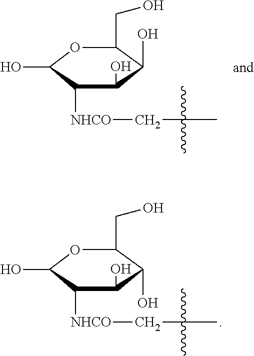

In a specific embodiment of the present invention, PEG is a multi-arm PEG residue, which has the structure shown in Formula VI below:

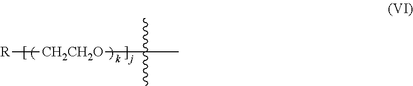

wherein, k independently an integer of 1-760, and preferably 70-380,

j is an integer of 3-8,

R is a core molecule of multi-arm PEG, selected from a residue of pentaerythrite, oligomeric pentaerythritol, methyl glucoside, sucrose, diethylene glycol, glycerol and polyglycerol.

Preferably, R is a residue of pentaerythrite, oligomeric pentaerythritol, glycerol and polyglycerol, more preferably selected from a residue of pentaerythrite, dipentaerythritol, tripentaerythritol, glycerol and hexaglycerol.

In one embodiment of the present invention, the multi-arm PEG includes only one linking site, with the structure below:

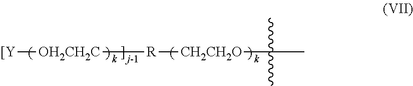

wherein, Y has the structure defined by foregoing Formula II of the present invention, preferably selected from methyl, ethyl, isopropyl, cyclopropyl, cyclopropylmethyl, cyclobutyl, cyclohexyl, benzyl,

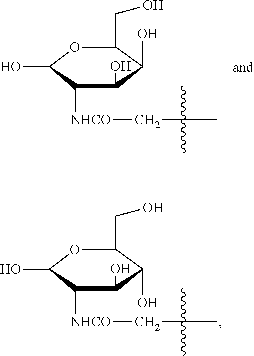

more preferably selected methyl,

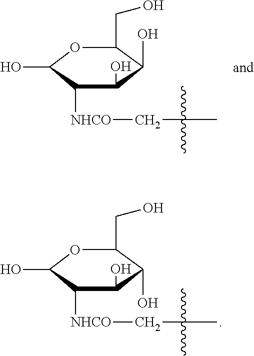

In one embodiment of the present invention, the multi-arm PEG has the structure below:

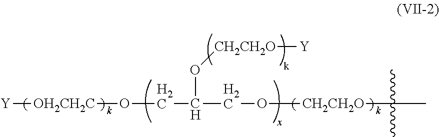

wherein, k independently an integer of 1-570, and preferably 55-285,

X is an integer of 1-10 (specifically for example, 1, 2, 3, 4, 5, 6, 7, 8, 9 or 10), more preferably an integer of 1-6,

wherein, Y has the structure defined by foregoing Formula II of the present invention, preferably selected from methyl, ethyl, isopropyl, cyclopropyl, cyclopropylmethyl, cyclobutyl, cyclohexyl, benzyl,

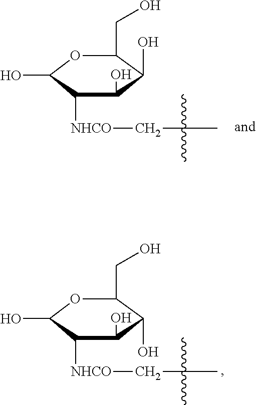

more preferably selected methyl,

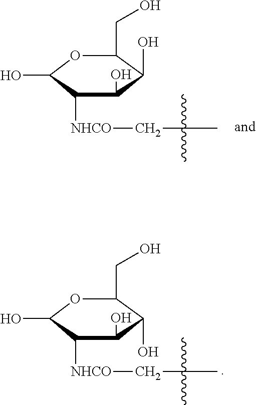

In another preferred embodiment of the present invention, the multi-arm PEG has the structure below:

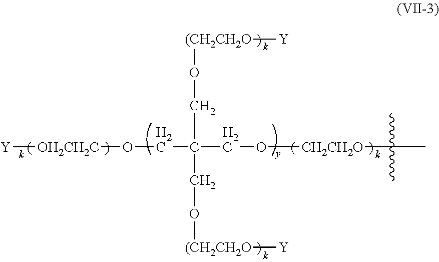

wherein, k independently an integer of 1-280, and preferably 28-140,

y is an integer of 1-10 (specifically for example, 1, 2, 3, 4, 5, 6, 7, 8, 9 or 10), preferably an integer of 1-5, and more preferably an integer of 1-3,

Y has the structure defined by foregoing Formula II of the present invention, preferably selected from methyl, ethyl, isopropyl, cyclopropyl, cyclopropylmethyl, cyclobutyl, cyclohexyl, benzyl,

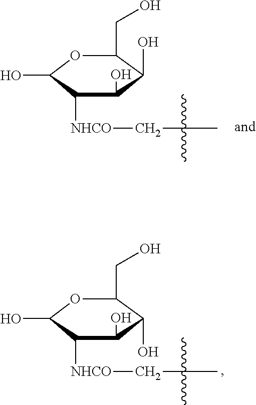

more preferably selected methyl,

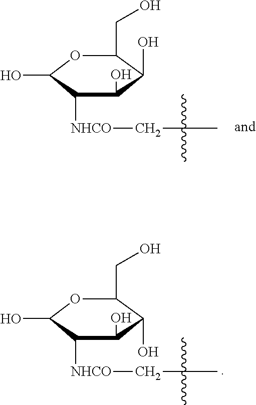

In the present invention, when PEG has the structure shown in Formula II, IV or V, one PEG molecule can link a drug D through a linking group X at the linking site thereof; when PEG has the structure shown in Formula III or VI, one PEG molecule has more than two linking sites, and can link the drug D through the linking group X at one of the linking sites, and under such circumstance, the conjugate includes only one drug residue. PEG can further link the drug D through the linking group X at a plurality of linking sites, and under such circumstance, the conjugate includes more than two drug residues. Preferably, when PEG is a PEG with more than two linking sites shown in Formula III or VI, PEG links the drug D through the linking group X at one of the linking sites, and other linking sites can link the capping terminal group Y (for example, methyl), for example the structure shown in Formula VII.

Preferably, in the conjugate, D has the structure below:

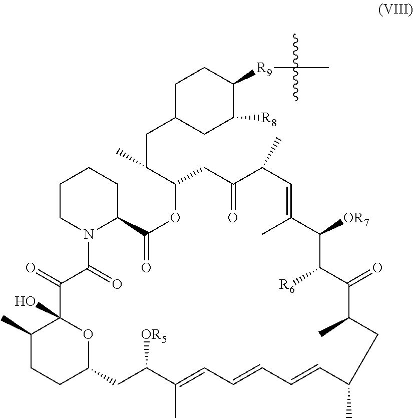

wherein,

R₅ is selected from H, C1-C6 alkyl, C2-C6 alkenyl, C6-C12 aryl and C7-C14 aralkyl;

R₆ is selected from H, hydroxyl and C1-C6 alkoxy;

R₇ is selected from H, C1-C6 alkyl, C2-C6 alkenyl, C6-C12 aryl, C7-C14 aralkyl and —C(O)R₇₁, wherein R₇₁ is selected from H, C1-C6 alkyl, C2-C6 alkenyl, C6-C12 aryl and C7-C14 aralkyl;

R₈ is selected from H, hydroxyl and C1-C6 alkoxy;

R₉ is selected from —O—, —O—R₅₁—O—,

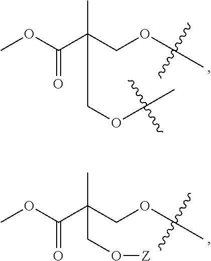

wherein R₉₁ is C1-C6 alkylene; and,

Z is H or a hydroxyl-protecting group.

Preferably, in Formula VIII, R₅ is H or C1-C6 alkyl, more preferably H, methyl or ethyl; and in one embodiment of the present invention, R₅ is methyl.

Preferably, in Formula VIII, R₆ is selected from H, hydroxyl and C1-C3 alkyl, more preferably is —OCH₃, —OCH₂CH₃ or —O(CH₂)₂CH₃; and in one embodiment of the present invention, R₆ is methoxyl.

Preferably, in Formula VIII, R₇ is selected from H, C₁-C₆ linear alkyl, C₂-C₁₀ linear alkenyl, phenyl, halogenated phenyl, benzyl, phenemyl and —C(O)R₇₁, wherein R₇₁ is selected from H and C₁-C₆ linear alkyl, more preferably selected from H, —OCH₃, —OCH₂CH₃ and —O(CH₂)₂CH₃; and in one embodiment of the present invention, R₇ is H.

Preferably, in Formula VIII, R₈ is selected from H, hydroxyl and C1-C3 alkyl, more preferably is —OCH₃, —OCH₂CH₃ or —O(CH₂)₂CH₃; and in one embodiment of the present invention, R₈ is methoxyl.

Preferably, R₉₁ is C1-C3 alkylene, for example, —CH₂—, —CH₂CH₂— and —CH₂CH₂CH₂—.

In a preferred embodiment of the present invention, in Formula VIII, R₅ is methyl; R₆ is methoxyl; R₇ is H; and R₈ is methoxyl.

Preferably, in the conjugate, D is selected from a residue of one of rapamycin, everolimus, temsirolimus, biolimusA9, ABT-578 and ridaforolimus.

More preferably, in the conjugate, D is selected from one of

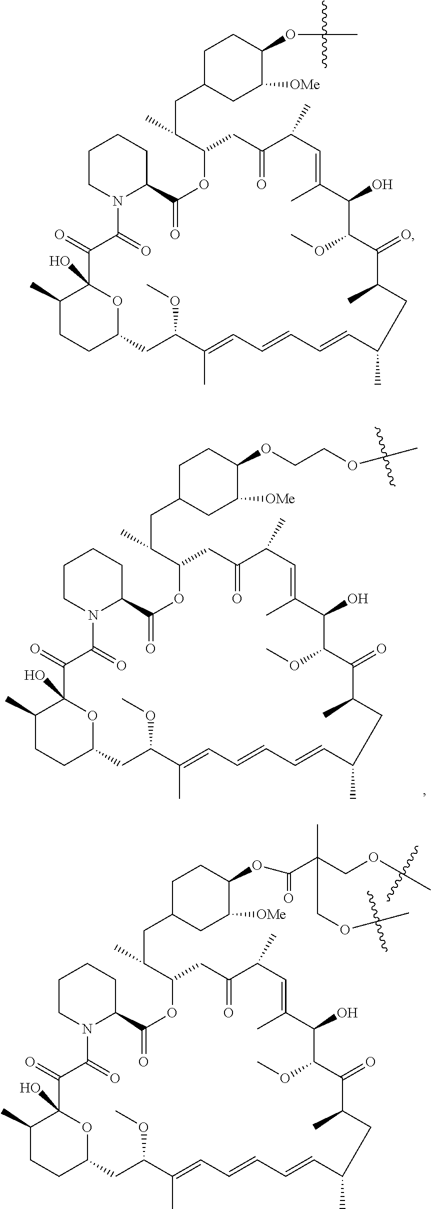

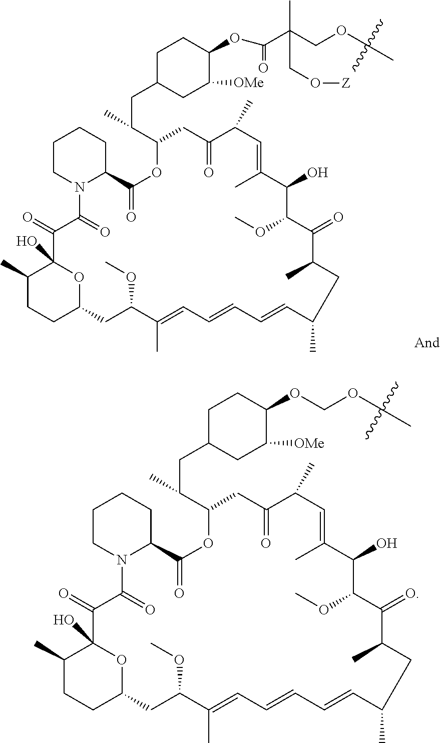

wherein, Z is H or a hydroxyl-protecting group.

For the hydroxyl-protecting group herein, a skilled in the art can select a frequently used hydroxyl-protecting group in the art upon actual situations on the basis of the present invention, for example, —CH₃, —C(CH₃)₃, —CH₂OCH₃, —COCH₃, —COC(CH₃)₃, —CH₂CH═CH₂, —Si(CH₃)₃,

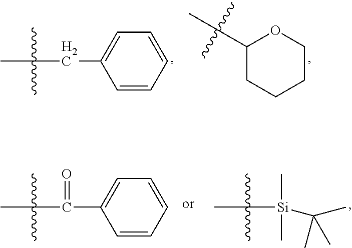

which is not specifically defined herein.

In a preferred embodiment of the present invention, in the conjugate, D has the structure below:

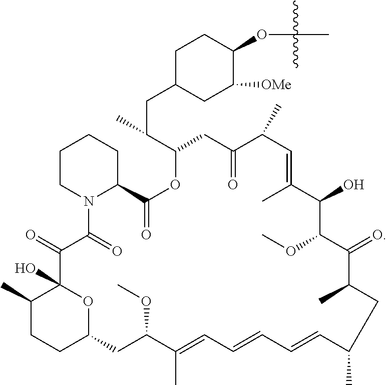 [SMILES: *O[C@@H]1CCC(C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

In specific embodiment of the present invention, the conjugate has the structure below:

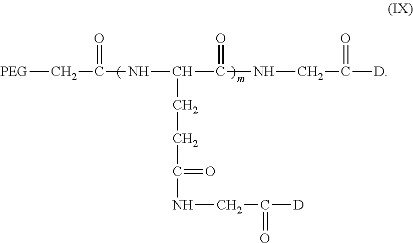

In the above Formula, PEG, m and D respectively have foregoing definitions of the present invention.

Preferably, in Formula IX, PEG has the structure of the foregoing Formula II; more preferably, PEG has the structure below:

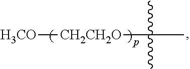

wherein p has the foregoing definition of the present invention.

Preferably, in Formula IX, the molecular weight of PEG is 10-50 KDa, more preferably 20 KDa.

Preferably, in Formula IX, m is 2 or 3.

Preferably, in Formula IX, D has the structure below:

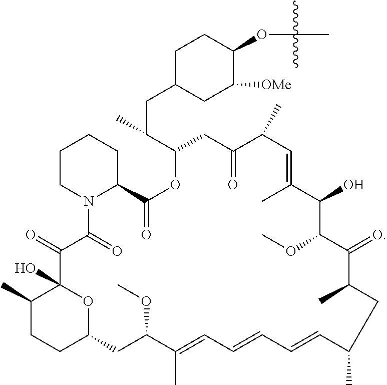 [SMILES: *O[C@@H]1CCC(C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

According to one aspect of the present invention, the immune response is caused by foreign immunogen; preferably, the use is the use of the conjugate of PEG and rapamycin in preparation of a medicament for reducing excessive immune responses of the subject to the foreign immunogen.

Preferably, the subject is mammal, preferably primates, more preferably human being.

Preferably, the foreign immunogen is selected from therapeutic agents, implants, artificial antigens, toxins, etc.

In one embodiment of the present invention, the foreign immunogen is the therapeutic agent; preferably, the therapeutic agent is a therapeutic macromolecule, more preferably a therapeutic protein, for example, cytokine, kuman hemoglobin, blood factor or blood coagulation factor, vascular endothelial growth factor antibody antagonist, hormone, antibody, enzyme and coenzyme; further preferably, the therapeutic protein is an antibody or enzyme and cozyme.

In the present invention, examples of the cytokine include, but are not limited: interleukin, colony stimulating factor, interferon, growth factor, tumor necrosis factor, and transforming growth factor beta family or chemokine family.

In the present invention, examples of the growth factor include, but are not limited to: adrenomedulin (AM), angiogenin (Ang), autocrine motility factor, bone morphogenetic protein (BMP), brain-derived neurotrophic factor (BDNF), epidermal growth factor (EGF), erythropoietin (EPO), fibroblast growth factor (FFGF), glialcellline-derivedneurotrophicfactor, glial cell line-derived neurotrophic factor (GDNF), granulocyte colony-stimulating factor (G-CSF), granulocyte-macrophage colony stimulating factor (GM-CSF), growth differentiation factor-9 (GDF9), hepatocyte growth factor (HGF), hepatocellular-derived growth factor (HDGF), insulin-like growth factor (IGF), migration-stimulating factor, myostatin (GDF-8), nerve growth factor (NGF) and other neurotrophic factor, platelet-derived growth factor (PDGF), thrombopoietin (TPO), transforming growth factor α (TGF-α), transforming growth factor β (TGF-β), tumor necrosis factor α (TNF-α), vascular endothelial growth factor (VEGF), Wnt signalling pathway, and placenta growth factor (P1GF), IL-1, IL-2, IL-3, IL-4, IL-5, IL-6 and IL-7.

In the present invention, the examples of the blood factor or blood coagulation factor includes, but are not limited to: factor I, factor II, tissue factor, factor V, factor VII (for example kong-acting coagulation factor VII), factor VIII, factor IX, factor X, factor Xa, factor XII, factor XIII, recombinant coagulation factor (for example recombinant coagulation factor VIII), von Willebrand factor, prekallikrein, high molecular weight kininogen, fibronectin, antithrombin III, albumin II, protein C, protein S, protein Z, protein Z-related protease inhibitor (ZPI), plasminogen, α-2-antiplasmin, tissue plasminogen activator (tPA), urokinase, plasminogen activator inhibitor-1 (PAI1), plasminogen activator inhibitor-2 (PAI2), cancer procoagulant and epoetin Alfa.

In one embodiment of the present invention, the blood factor or blood coagulation factor is factor VIII.

In the present invention, the hormone includes but is not limited to: melatonin (N-acetyl-5-methoxytryptamine), serotonin, thyroxine (or tetraiodothyronine) (thyroid hormone), triiodothyronine (thyroid hormone), adrenalin (or adrenal hormone), norepinephrine (or norepinephrine hormone), dopaminem (or prolactin inhibiting hormone), anti-Mullerian hormone (or Mullerian regression factor or hormone), adiponectin, adrenocorticotrophic hormone (or corticotrophin), proangiotensin and angiotonin, antidiuretic hormone (or vasopressin, arginine vasopressin), atrial natriuretic peptide (or atrial natriuretic factor (atriopeptin), calcitonin, cholecystokinin, corticotropin-releasing hormone, erythropoietin, follicle stimulating hormone, gastrin, growth hormone releasing peptide, glucagon, glicetin (GLP-1), GIP, gonadotropin-releasing hormone, growth hormone releasing hormone, human chorionic gonadotropin, chorionic somatomammotropin, growth hormone, inhibin, insulin, insulin-like growth factor (or somatomedins), leptin, leutinizing hormone, melanocyte stimulating hormone, orexin, oxytocin, parathyroid hormone, prolactin, relaxin, secretin, somatostatin, thrombopoietin, thyroid-stimulating hormone (or thyrotropin), thyrotropin releasing hormone, cortisol, aldosterone, testosterone, dehydroisoandrosterone, androstendione, dihydrotestosterone, estrone, estriol, progestin, calcitriol (1, 25 dihydroxy vitamin D), calcifediol (25-hydroxy vitamin D), prostanoid, leukotrienes, prostacyclin, thromboxanes, prolactin releasing hormone, lipotrophin, brain natriuretic peptide, neusopeptide Y, histamine, endothelin, pancreatic polypeptide, renin, enkephalin, etc.

OmalizumabIn the present invention, the antibody comprises a monoclonal antibody, a polyclonal antibody, a dipolymer, a polymer, a bispecific antibody, a polyspecific antibody and an antibody fragment (for example, Fab, Fab′, F(ab)₂, F(ab′)₂, Fv, etc.). Examples of the monoclonal antibody specifically include: adalimumab (HUMIRA), tocilizumab, ocrelizumab, belimumab, infliximab, rituximab, trastuzumab, bevacizumab, alemtuzumab, cetuximab, panitumumab, nimotuzumab, pertuzumab, ibritumomab, tositumomab, omalizumab, abagovomab, abciximab, adecatumumab, afelimomab, afutuzumab, alacizumab pegol, altumomabpentetate, anatumomabmafenatox, anrukinzumab)(IMA-638), apolizumab, arcitumomab, aselizumab, atorolimumab, bapineuzumab, basiliximab, bavituximab, bectumomab, belimumab, bertilimumab, besilesomab, bevacizumab, biciromab, bivatuzumabmertansine, blinatumomab, brentuximabvedotin, ABT-874 (briakinumab), canakinumab, cantuzumabmertansine, capromabpendetide, catumaxomab, cedelizumab, certolizumabpegol, citatuzumabbogatox, cixutumumab, clenoliximab, clivatuzumabtetraxetan, CNTO148(golimumab), CNTO1275 (ustekinumab), conatumumab, dacetuzumab, daclizumab, denosumab, detumomab, dorlimomabaritox, dorlixizumab, ecromeximab, eculizumab, edobacomab, edrecolomab, efalizumab, efungumab, elsilimomab, enlimomabpegol, epitumomabcituxetan, epratuzumab, erlizumab, ertumaxomab, etaracizumab, exbivirumab, fanolesomab, faralimomab, felvizumab, fezakinumab, figitumumab, fontolizumab, foravirumab, fresolimumab, galiximab, gantenerumab, gavilimomab, gemtuzumabozogamicin, golimumab, gomiliximab, ibalizumab, ibritumomabtiuxetan, igovomab, imciromab, infliximab, intetumumab, inolimomab, inotuzumabozogamicin, ipilimumab, iratumumab, keliximab, labetuzumab, lebrikizumab, lemalesomab, lerdelimumab, lexatumumab, libivirumab, lintuzumab, lucatumumab, lumiliximab, mapatumumab, maslimomab, matuzumab, mepolizumab, metelimumab, milatuzumab, minretumomab, mitumomab, morolimumab, motavizumab, muromonab-CD3, MYO-029 (stamulumab), nacolomabtafenatox, naptumomabestafenatox, natalizumab, nebacumab, necitumumab, nerelimomab, nimotuzumab, nofetumomabmerpentan, ocrelizumab, odulimomab, ofatumumab, oportuzumabmonatox, oregovomab, otelixizumab, pagibaximab, palivizumab, panitumumab, panobacumab, pascolizumab, pemtumomab, pertuzumab, pexelizumab, pintumomab, priliximab, pritumumab, PRO140, rafivirumab, ramucirumab, ranibizumab, raxibacumab, regavirumab, reslizumab, rilotumumab, robatumumab, rontalizumab, rovelizumab, ruplizumab, satumomab, sevirumab, sibrotuzumab, sifalimumab, siltuximab, siplizumab, solanezumab, ASONEP(sonepcizumab), sontuzumab, stamulumab, sulesomab, tacatuzumabtetraxetan, tadocizumab, talizumab, tanezumab, taplitumomabpaptox, tefibazumab, telimomabaritox, tenatumomab, teneliximab, teplizumab, tGN1412, ticilimumab, tremelimumab, tigatuzumab, TNX-355 (ibalizumab), TNX-650, TNX-901 (talizumab), toralizumab, tositumomab, tremelimumab, tucotuzumabcelmoleukin, tuvirumab, urtoxazumab, ustekinumab, vapaliximab, vedolizumab, veltuzumab, vepalimomab, visilizumab, volociximab, votumumab, zalutumumab, zanolimumab,ziralimumab, zolimomabaritox, etc.

In one embodiment of the present invention, the monoclonal antibody is adalimumab.

In the present invention, the enzyme and coenzyme includes, but is not limited to: immunoenzymes, microbial enzymes, redox enzymes, transferring enzymes, hydrolytic enzymes, lyases, isomerases or ligase, etc., and specifically includes imiglucerase, α-galactosidase A (α-galA), agalsidase β, acid α-glucosidase (GAA), recombinant glucosinolate α (LUMIZYME), α-glucosidase, phenol sulfatase, aldurazyme, elaprase, galsulfase (NAGLAZYME), PEGylated recombinant uricase (KRYSTEXXA), PEGylated recombinant Candida uricase, L-Asparaginasum, etc.

In one embodiment of the present invention, the enzyme is α-galactosidase A (α-galA), acid α-glucosidase (GAA), PEGylated recombinant uricase (KRYSTEXXA), PEGylated recombinant Candida uricase or L-Asparaginasum.

In another embodiment of the present invention, the foreign immunogen is an artificial antigen carrier; the artificial antigen carrier refers to a molecule with immunogenicity that is linked (for example, coupling) with a hapten or another antigen as a carrier to increase the immunogenicity thereof to form an artificial antigen, including proteins, polypeptide polymers, macromolecular polymers and some particles. The carrier of the protein (also referred to carrier protein) includes, but is not limited to: keyhole limpet hemoeyanin (KLH), bovine serum albumin (BSA), human serum albumin (HSA), ovalbumin (OVA), etc. The polypeptide polymer carrier includes polylysine (PLL), etc. The particle carrier includes some bacterial particles, viruses, etc.

In a specific embodiment of the present invention, the artificial antigen carrier is a protein carrier.

In one embodiment of the present invention, the protein carrier is keyhole limpet hemoeyanin (KLH).

In another embodiment of the present invention, the foreign immunogen is an implant; preferably, the implant is a cell, tissue, organ or other biologically appropriate implant for transplantation; and the implant can be an autologous or xenogenic (for example, individuals of the same species but different in genetype) cell, tissue and organ, and can further be an artificial material (for example, an artificial bone, an artificial trachea), etc.

In a specific embodiment of the present invention, the implant is a cell, preferably a stem cell, for example, hemocytoblast, neural stem cell, etc.

In a specific embodiment of the present invention, the implant is a tissue, preferably selected from skins, bones and chondrogenic differentiation, dermis and fat, mucous membranes, anadesma, cornea, muscles, nerves, artificial materials, etc.

In a specific embodiment of the present invention, the implant is an organ, preferably selected from heart, pancreas, kidneys, lungs, livers, etc.

Preferably, in use of the present invention, the conjugate is used before or after the foreign immunogen is used or used together with the foreign immunogen.

Other exemplary therapeutic agents such as chemical therapeutic agents include, but are not limited to: anti-metabolic drugs, platinum-based agents, alkylating agents, tyrosine kinase inhibitors, anthracycline antibiotics, vinca alkaloids, proteasome inhibitors, topoisomerase inhinitors, etc. Those therapeutic agents are known by a skilled in the art. The present invention is not limited in this aspect.

The present invention further provides use a conjugate of PEG (polyethylene glycol) and rapamycin in preparation of a medicament for strengthening the effect of a first medicament, wherein the first medicament is selected from at least one of cytokine, human hemoglobin, blood factor or blood coagulation factor, vascular endothelial growth factor antibody antagonist, hormone, antibody, enzyme and coenzyme.

Preferably, the first medicament is for treating tumors, hematologic diseases, infectious diseases, immune system diseases or metabolic diseases.

More preferably, the tumors are selected from lung cancer, kidney cancer, melanoma, liver cancer, head and neck cancer, skin cancer, squamous-cell carcinoma, ovarian cancer, bone cancer, colorectal cancer, bladder cancer, stomach cancer, pancreatic cancer, prostate cancer, Hodgkin lymphoma, follicular lymphoma, chronic or acute leukemia, mesothelioma, pancreatic cancer, breast cancer, multiple myeloma and other tumors; the infectious diseases are selected from diseases caused by HIV infection, diseases caused by hepatitis virus (A, B and C) infection, diseases caused by herpes virus infection and diseases caused by influenza virus infection; the immune system diseases are selected from lupus erythematosus, rheumatoid arthritis, ankylosing spondylitis, asthenic bulbar paralysis, multiple sclerosis, autoimmune haemolytic anaemia, autoimmune hepatitis, scleroderma, polyarteritis nodose and Wegener granuloma; the genetic diseases are selected from Fabry disease, Pompe disease, lysosomal storage diseases, genetic muscle diseases and genetic metabolic diseases; and the metabolic diseases are selected from disorders of amino acid metabolism (for example, branched-chain-related diseases, hyperaminoacidemia, hyper aminoaciduria), urea metabolism disorders, hyperammonemia, mucopolysaccharidosis, accumulation diseases (for example glycogen-storage disease and lipoidosis), glycogen-storage disease I (for example Cushing disease), malabsorption (for example intestinal carbohydrate malabsorption), oligosaccharidase deficiency (for example maltase deficiency, lactase deficiency and sucrase deficiency), fructose metabolism disorders, galactose metabolism disorders, galactosemia, carbohydrate metabolism disorders (for example diabetes), hypoglycemia, pyruvate metabolism disorders, hypolipidemia, hypolipoproteinemia, hyperlipemia, hyperlipoprotememia, carnitine or acylcarnitine translocase deficiency, porphyrin metabolism disorders, disorders of porphyrin and purine metabolism, lysosomal disease, nervous and nervous system diseases, sulfatide lipidosis, leukoencephalopathy and Lesh-nain syndrome.

The present invention further provides use of the conjugate of PEG and apamycin in preparation of a medicament for improving the effect of a gene therapy.

Preferably, the gene therapy is a non-immunogenic gene therapy, for example a non-immunogenic gene therapy which uses virus vectors.

More preferably, the gene therapy is is for treating cancers, hematologic diseases, infectious diseases, immune system diseases or metabolic diseases.

Further preferably, the tumors are selected from lung cancer, kidney cancer, melanoma, liver cancer, head and neck cancer, skin cancer, squamous-cell carcinoma, ovarian cancer, bone cancer, colorectal cancer, bladder cancer, stomach cancer, pancreatic cancer, prostate cancer, Hodgkin lymphoma, follicular lymphoma, chronic or acute leukemia, mesothelioma, pancreatic cancer, breast cancer, multiple myeloma and other tumors; the infectious diseases are selected from diseases caused by HIV infection, diseases caused by hepatitis virus (A, B and C) infection, diseases caused by herpes virus infection and diseases caused by influenza virus infection; the immune system diseases are selected from lupus erythematosus, rheumatoid arthritis, ankylosing spondylitis, asthenic bulbar paralysis, multiple sclerosis, autoimmune haemolytic anaemia, autoimmune hepatitis, scleroderma, polyarteritis nodose and Wegener granuloma; the genetic diseases are selected from Fabry disease, Pompe disease, lysosomal storage diseases, genetic muscle diseases and genetic metabolic diseases; and the metabolic diseases are selected from disorders of amino acid metabolism (for example, branched-chain-related diseases, hyperaminoacidemia, hyper aminoaciduria), urea metabolism disorders, hyperammonemia, mucopolysaccharidosis, accumulation diseases (for example glycogen-storage disease and lipoidosis), glycogen-storage disease I (for example Cushing disease), malabsorption (for example intestinal carbohydrate malabsorption), oligosaccharidase deficiency (for example maltase deficiency, lactase deficiency and sucrase deficiency), fructose metabolism disorders, galactose metabolism disorders, galactosemia, carbohydrate metabolism disorders (for example diabetes), hypoglycemia, pyruvate metabolism disorders, hypolipidemia, hypolipoproteinemia, hyperlipemia, hyperlipoprotememia, carnitine or acylcarnitine translocase deficiency, porphyrin metabolism disorders, disorders of porphyrin and purine metabolism, lysosomal disease, nervous and nervous system diseases, sulfatide lipidosis, leukoencephalopathy and Lesh-nain syndrome.

The present invention provides use of the conjugate of PEG (polyethylene glycol) and rapamycin in reduction of immune response.

The present invention further provides use of the conjugate of PEG and apamycin in improvement on the therapeutic effect of a second medicament.

The present invention further provides use of the conjugate of PEG and apamycin in improvement on the effect of a gene therapy.

The present invention further provides a pharmaceutical composition, wherein the pharmaceutical composition comprises the first medicament and the conjugate of PEG and rapamycin of the present invention, and the first medicament is selected from at least one of cytokine, human hemoglobin, blood factor or blood coagulation factor, vascular endothelial growth factor antibody antagonist, hormone, antibody, enzyme and coenzyme.

The medicament or pharmaceutical composition of the present invention is in form of tablet, pill, powder, pastille, pulvis, elixir, suspension, lotion, solution, syrup, aerosol, ointment, patch, soft capsule, hard capsule, suppository, sterile injection solution and lyophilized powder. Preferably, the medicament or pharmaceutical composition of the present invention is sterile injection solution and lyophilized powder.

The medicament or pharmaceutical composition of the present invention further includes pharmaceutically acceptable adjuvants, wherein the adjuvants are selected from carriers, diluent (one or more than two of a group consisting of lactose, glucose, sucrose, sorbitol, mannitol, starch, arabic gum, calcium phosphate, alginate, bassora gum, gelatin, calcium silicate, microcrystalline cellulose, polyvinyl pyrrolidone, cellulose, water, syrup, methylcellulose, methyl hydroxybenzoate and propyl hydroxybenzoate, talcum, magnesium stearate and mineral oil), lubricant, solubilizer, wetting agent, emulsifier, suspending agent, antiseptic, and edulcorant or correctant.

The administration method of the medicament or pharmaceutical composition of the present invention is selected from oral, subcutaneous, intracutaneous, intramuscular, endoperitoneal, intravenous, nasal, epidural, sublingual, nasal, intracephalic, intravaginal, diadermic and rectal administration.

The present invention provides a conjugate of PEG (polyethylene glycol) and rapamycin and use thereof, in particular use in preparation of a medicament for reducing immune response (in particular for reducing excessive immune responses caused by foreign immunogen such as a therapeutic agent and an implant), wherein conjugate of PEG and rapamycin can remarkably lower the generation rate of an antibody directed to foreign immunogen such as the therapeutic agent and the implant, and reduce excessive immune responses caused thereby. The conjugate of PEG and rapamycin has beneficial effects of ensuring and even improving the treatment effect of a therapeutic agent, improving the own immunity of a subject, reducing and even eliminating graft rejection, and is advantageous in a relatively simple preparation process thereof, low cost, easy industrial production, and a high application value.

### DETAILED DESCRIPTION OF THE INVENTION

Unless otherwise specified, all scientific and technological terms used in the present invention have meanings the same as the terms generally understood by a skilled in the art. For example:

“Alkyl” refers to a linear or branched hydrocarbon radical without an unsaturated bond. C1-C6 alkyl refers to an alkyl group containing 1-6 carbon atoms, such as methyl, ethyl, n-propyl, isopropyl, n-butyl, isobutyl, tertiary-butyl, n-amyl, n-hexyl, etc. If an alkyl group is replaced by a cycloalkyl group, a corresponding “cycloalkylalkyl” radical is obtained. C4-10 cycloalkylalkyl refers to a cycloalkylalkyl group containing 4-10 carbon atoms, such as cyclopropylmethyl, cyclopropylethyl, cyclobutylmethyl, cyclopentylmethyl, cyclohexylmethyl, etc. If an alkyl group is replaced by an aryl group, a corresponding “aralkyl” radical is obtained. C7-14 aralkyl refers to an aralkyl group containing 7-14 carbon atoms, such as benzyl, benzhydryl or phenemyl.

“Alkoxy” refers to a substituent group formed after the hydrogen in the hydroxyl is substituted by alkyl. C1-C6 alkoxy refers to an alkoxy group containing 1-6 carbon atoms, such as methoxyl, oxethyl, oxypropyl, butoxy, etc.

“Cycloalkyl” refers to an alicyclic hydrocarbon, containing for example 1-4 single rings and/or fused rings, and containing 3-18 carbon atoms, preferably 3-10 carbon toms, such as cyclopropyl, cyclohexyl or adamantyl. In the present invention, “C3-6 cycloalkyl” in refers to a cycloalkyl group containing 3-6 carbon atoms, such as cyclopropyl, cyclobutyl, cyclopentyl and cyclohexyl.

“Alkyl” refers to a monocyclic or polycyclic radical, including polycyclic radicals containing monoaryl groups and/or fused aryl groups, for example containing 1-3 single rings or fused rings and 6-18 carbon atoms. In the present invention, C6-10 aryl refers to an aryl group containing 6-10 carbocyclic atoms, such as phenyl and naphthyl.

“Heterocyclic group” refers to a heteroaromatic group or a heterocyclic group containing 1-3 single rings and/or fused rings and 3 to about 18 annular atoms. Preferably, each of the heteroaromatic group and the heterocyclic group contains 5 to about 10 annular atoms. In the present invention, the appropriate heterocyclic group in the compound contains 1, 2 or 3 types of heteroatoms, and the heteroatoms are selected from N, O or S atoms. Nitrogen heterocyclic ring refers to that the heteroatoms include N atoms.

“Monosaccharide” generally refers to a polyhydroxyl aldehyde or polyhydroxyl ketone containing 3-6 carbon atoms. According to the number of carbon atoms, monosaccharides can be classified into triose, tetrose, pentose, hexose, etc. Common monosaccharides include glucose, fructose, galactose, ribose, deoxyribose, etc.

“Oligosaccharide” refers to a compound polymerized by 2-10 glucosidic bonds. The glucosidic bond is formed by the hemithioacetal hydroxyl group of one oligosaccharide and a certaom hydroxyl group of another oligosaccharide through dehydration synthesis. Common oligosaccharides include disaccharides, trisaccharides, etc. “Disaccharide” refers to a compound formed by two monosaccharide molecules through the glucosidic bond, for example, ucrose, lactose, maltose, etc. “Trisaccharide” refers to a compound formed by three monosaccharide molecules through the glucosidic bonds, for example, gentianose, gossypose, etc.

Rapamycin is also referred to as sirolimus. In the present invention, derivatives of rapamycin include, but are not limited to compounds with structures and functions similar to those of rapamycin, for example, everolimus (RAD 001 (Novartis)), temsirolimus (CC1-779 Wyeth)), biolimus A9, ABT-578 (zotarolimus), deforolimus, ridaforolimus, AP23573, MK-8669, etc. Rapamycin and the derivatives thereof have respectively the chemical structures shown below:

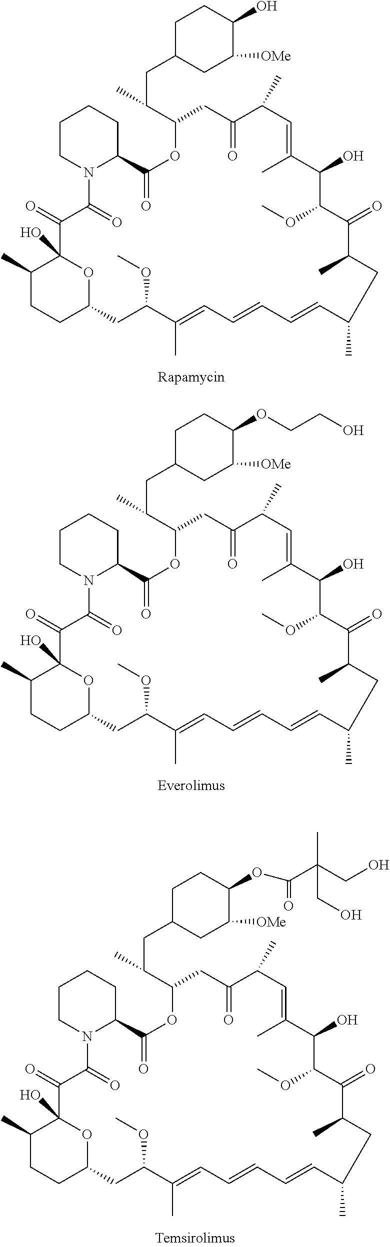

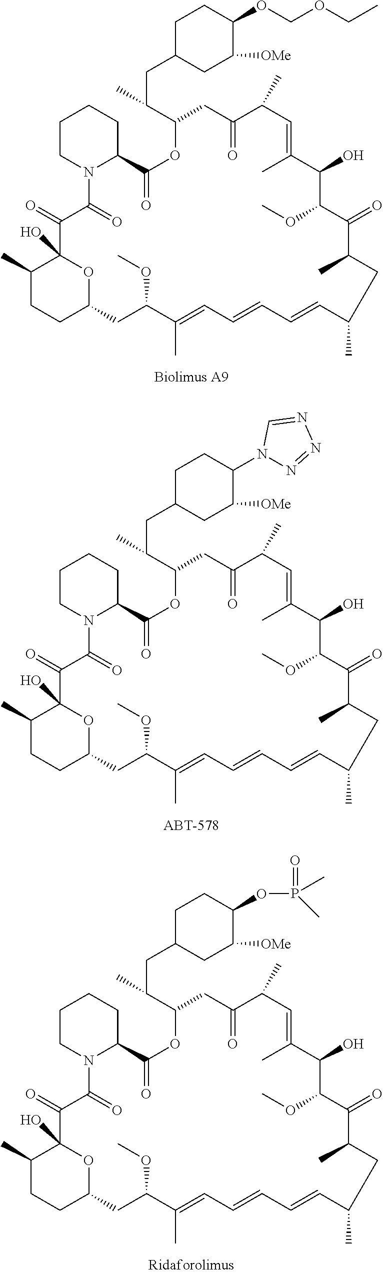

In the present invention, the “therapeutic agent” refers to a substance that can be given to the subject and has a therapeutic effect, for example, proteins, carbohydrates, lipids, nucleic acid and other macromolecular substances. In one embodiment of the present invention, the therapeutic agent is a therapeutic macromolecule. Subject's intake of the therapeutic macromolecule may cause excessive immune responses, for example, generation of a specific antibody against the therapeutic macromolecule. In a specific embodiment of the present invention, the therapeutic agent is a therapeutic protein.

In the present invention, the “excessive immune responses” are caused by exposure to the antigen to promote or aggregate the diseases, disorders or illnesses (or symptoms thereof) herein, or refer to the diseases, disorders or illnesses (or symptoms thereof) herein. Such immune responses usually have an adverse effect on the health of the subject or refer to symptoms of the adverse effect on the health of the subject. Excessive immune responses include generation of an antigen-specific antibody, the reproduction of antigen-specific B cells and/or the reproduction and/or activity of the active or antigen-specific CD4+T cells. Generally speaking, such excessive immune responses are specific for the therapeutic macromolecule and are beneficial effects which counteract with the adverse effects of use of the therapeutic macromolecule.

In the present invention, the term “antibody” is used by the broadest meaning thereof and particularly covers the monoclonal antibody, polyclonal antibody, dipolymer, polymer, polyspecific antibody (for example bispecific antibody) and antibody segment, as long as they present required biological activities (Miller et.al. (2003) Journal of Immunology, 170:4854-4861). The antibody can be a mice, human, humanized or chimeric antibody, or comes from other species. The antibody can be of any type (for example, IgG, IgE, IgM, IgD and IgA) and of any category (for example, IgG1, IgG2, IgG3, IgG4, IgA1 and IgA2).

In the present invention, the “subject” is mammal, including but not limited to: primates (for example, human beings, monkeys, apes, etc.), even-toed ungulates (for example, pigs, sheets, cattle, etc.), odd-toed ungulates (for example, horses), rodents (for example, mice, capybaras, etc.), meat-eating animals (including canidae (for example, wolves, dogs), felidae (for example tigers, cats), ursidae (for example, panda, etc.), preferably human beings.

In the present invention, “transplantation” refers to a process of transplanting corresponding healthy cell, tissue or organ into a body when a certain cell, tissue or organ of the body is injured to cause irreversible structural and functional lesions. Common transplantation includes autotransplantation, allotransplantation and homogeneic transplantation.

In the present invention, the “pharmaceutically acceptable adjuvant” is an ingredient which does not contain an impurity with biological activity or other adverse activity, for example, the ingredient can be incorporated into a public pharmaceutical preparation and given to patients, without causing remarkable adverse biological effects or interacting with other ingredients in the pharmaceutical preparation in an adverse way.

In the present invention, the “treatment” includes suppression, delay, alleviation, weakening, limitation, mitigation or elimination of diseases, disorders, illnesses or symptoms, and occurrence and/or progress, and/or symptoms thereof.

In the present invention, “comprise” and “include” are “open’ or “inclusive” terms, so that those terms include listed elements and additional and unmentioned elements.

In the present invention, the term “about” refers usually to +/−5% of the value, more usually to +/−4% of the value, further usually to +/−3% of the value, further usually to +/−2% of the value, further usually to +/−1% of the value, and further usually to +/−0.5% of the value.

The following clearly and completely describe the technical solution of the present invention in conjunction with the embodiments of the present invention. Apparently, the described embodiments are merely a part of the embodiment of the present invention, but not all embodiments. Based on the embodiments in the present invention, all other embodiments obtained by an ordinarily skilled person in the art without creative labor shall fall within the protective scope of the present invention.

### Examples

The rapamycin used in the embodiments were purchased from Wuhan Yuancheng Gongchuang Science and Technology Co., Ltd. Monomethoxy polyethylene glycol-glutamic dipeptide and glycine rapamycin ester were provided by Tianjin Jemkem Technology Co., Ltd. Others were commercially available reagents. Experimental animals were Balb/c female mice, purchased from Beijing Huafu Kang Biological Technology Co., Ltd.; the immunogen was keyhole limpet hemoeyanin (KLH), purchased from an American company Sigma.

### Example 1 Preparation of the Conjugate (JK1208R) of Monomethoxy Polyethylene Glycol (Number-Average Molecular Weight of 20000)-Glutamic Dipeptide-Rapamycin (with the Structural Formula Below)

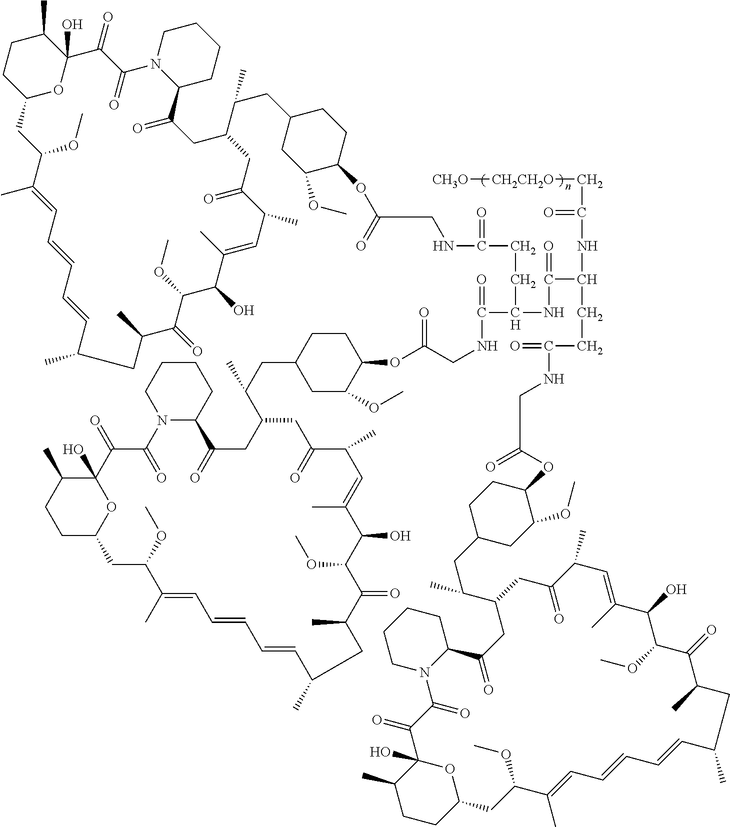

Monomethoxy polyethylene glycol-glutamic dipeptide (20 KDa, 5 g, 0.25 mmol), glycine rapamycin ester (486 mg, 0.5 mmol), HOBt (34 mg, 0.25 mmol) and DMAP (61 mg, 0.5 mmol) were added into a reaction bottle. Dichloromethane was used to dissolve them, and the system was cooled in an ice bath. Then, DCC (155 mg, 0.75 mmol) was dropped into the solution containing dichloromethane. After drop, the system was naturally warmed to room temperature to react overnight. On the second day, the reaction liquid was concentrated. The residue was crystalized using isopropanol to obtain 4.7 g conjugate (JK1208R) of monomethoxy polyethylene glycol-glutamic dipeptide-rapamycin (n was about 450).

### Example 2 Evaluation of Influences on the Keyhole Limpet Hemoeyanin (KLH) Antibody Level

### 2.1 Experimental Animals

Species: mouse

Strain: Balb/c

Grade: SPF

Breeding institution: Beijing Huafu Kang Biological Technology Co., Ltd.

Weeks of age when purchased: 5

Weight when purchased: 14-16 g

### 2.2 Breeding Conditions of the Experimental Animals

The experiment began 3 days after the animals were bred in the experimental environment. The animals were bred in the IVC (Independent Ventilation Cage) in the SPF-grade animal room, 4 in each age. The label on each cage marked the number, gender, strain, date of arrival, administration solution, experimental number, group number and beginning date of experiment of the animals therein. All cages pads, feed and drinking water were sterilized before use. The cages, feed and drinking water were replaced twice a week. The breeding environment and lighting conditions were as follows:

Temperature: 20-26° C.

Humidity: 40-70%

Lighting cycle: 12 h lighting, and 12 h without lighting (Lighted up at 8:00 a.m., and lighted off at 8:00 p.m.)

### 2.3 Instruments and Consumables

### 2.3.1 Main Reagents and Consumables

| Reagents and consumables used | Manufacturer |
|---|---|
| Freund's complete adjuvant | Sigma's Cat. No F5881 |
| Freund's incomplete adjuvant | Sigma's Cat. No F5506 |

### 2.3.2 Main Instruments

| Instruments used | Manufacturer |
|---|---|
| DEM-3 Automatic | Beijing Tuopu Analytical |
| microplate washer | Instrument Co. Ltd. |
| Microplate reader | BIO-RAD 680 |

### 2.4 Experiment Method and Steps

### 2.4.1. Antigenic Information: Immunogen: KLH; Tested Substance: KLH

### 2.4.2 Test Process

Group G1 (Model Group)

Animal Immunization

Primary immunization: On the first day, 100 ug antigen (immunogen) and Freund's complete adjuvant in the same volume were formed into a water-in-oil compound by means of mutual injection with a syringe, and then the compound was given to each of 18 mice by subcutaneous multi-point injection.

Second immunization: On the 14th day, 100 ug antigen (immunogen) and Freund's incomplete adjuvant in the same volume were formed into a water-in-oil compound by means of mutual injection with a syringe, and then the compound was given to each of 18 mice by subcutaneous multi-point injection.

Third immunization: On the 24th day, 100 ug antigen (immunogen) and Freund's incomplete adjuvant in the same volume were formed into a water-in-oil compound by means of mutual injection with a syringe, and then the compound was given to each of 18 mice by subcutaneous multi-point injection.

Test of tail blood: On the 28th-29th day, about 20 ul tail blood was collected from the tail vein, followed by 30 min RT and 5 min 5000 rpm centrifugation; then haemocyte precipitate was removed; and serum was taken for Elisa potency assay.

Fourth immunization: On the 34th day, 100 ug antigen (immunogen) and Freund's incomplete adjuvant in the same volume were formed into a water-in-oil compound by means of mutual injection with a syringe, and then the compound was given to each of 18 mice by subcutaneous multi-point injection.

Group G2 (Group IV): immunization was performed with reference to group G1; moreover, 620 mg JK1208R (prepared in Example 1) was given by intravenous injection on the 0th day and 7th day, respectively.

Group G2 (Group IV): immunization was performed with reference to group G1; moreover, 52 μg rapamycin was given by oral administration on the 0th day and 7th day, respectively.

Test of tail blood: On the 40th-41st day, about 20 ul tail blood was collected from the tail vein, followed by 30 min RT and 5 min 5000 rpm centrifugation; then haemocyte precipitate was removed; and serum was taken for ELISA potency assay.

### 2.4.3 Potency Assay (Enzyme Linked Immunosorbent Assay (ELISA)

Coating: 10 ug antigen (tested substance) and 10 ml coating buffer (1.6 g NaCO₃+2.92 g NaHCO₃+100 ml deionized water) were mixed; the solution was applied to the Elisa microplate, 100 ul per pore; the microplate was incubated for 2 h at 37° C. or kept still overnight at 4° C.

Blocking: The antigen coating buffer was sucked and removed from the coated microplate; a blocking buffer (5% dried skimmed milk and coating buffer) was directly added, 150 ul per pore; the microplate was treated for 2 h at 37° C., followed by three times of washing with de-ionized water; then the microplate was provisionally stored at 4° C.

Assay: serum and 1×PBS was mixed at a ratio of 1:1000; 150 ul diluent was added into the first pore; 100 ul 1×PBS were respectively added into each of the 2nd-8th pores; 50 ul liquid was transferred from the first pore into the second pore, followed by suction blow for 10 times; then, 50 ul liquid was transferred into the third pore, followed by suction blow for 10 times; next, 50 ul liquid was transferred into the fourth pore, followed by suction blow for 10 times; the test was done in the similar way in turn until the 7th pore, 50 ul liquid was removed from the 7th pore; the Beth pore was a negative control group; the microplate was incubated for 1 h at 37° C., followed by three times of washing with de-ionized water; then a second antibody (goat-anti-mouse IgG-HRP 1:10000 1×PBS) was added, 100 ul each pore; the microplate was incubated for 30 min at 37° C., followed by three times of washing with de-ionized water; a chromogenic solution was added (according to the instructions on the reagent box), 100 ul each pore; the microplate was incubated for 10 min at 37° C. and then ended (2M sulphuric acid, 30 ul per pore). The OD value was tested with the microplate reader.

### 2.5 Experimental Result

The statistical potency data of the groups G1, G2 and G3 after the fourth immunization are shown in Table 1 and accompanying drawing 1.

TABLE 1
Influences of Group PO and Group IV on generation of HLK antibody (x̅ ± sd)

| | Data obtained | Data obtained | Data obtained | Data obtained |
|---|---|---|---|---|
| | after the third | after the third | after the fourth | after the fourth |
| | immunization | immunization | immunization | immunization |
| Group | (1: 1K) | (1: 3K) | (1: 1K) | (1: 3K) |
| Model | 1.65 ± 0.12 | 1.21 ± 0.11 | 2.58 ± 0.13 | 2.36 ± 0.27 |
| Group PO | 1.77 ± 0.33 | 1.49 ± 0.38 | 2.56 ± 0.11 | 2.40 ± 0.24 |
| Group IV | 1.33 ± 0.24 | 0.86 ± 0.20* | 1.85 ± 0.32*** | 1.54 ± 0.31*** |

Note:
Compared with the model group,
*P < 0.05,
**P < 0.01,
***P < 0.001.

From the results it can be known that, compared with the immune model group after the third immunization, the result of statistical data analysis at 1:3K shows that the Group IV and the immune model group show remarkably difference, while the group PO shows no remarkable difference. Compared with the immune model group after the fourth immunization, the result of statistical data analysis at 1:1K and 1:3K shows that the Group IV and the immune model group show remarkably difference, while the group PO shows no remarkable difference. This means that the Group IV could remarkably resist the generation of the KLH antibody after the fourth immunization.

The above describes merely preferable embodiments of the present invention and shall no limit the present invention. Any modifications, equivalent substitutions, improvements and the like made within the spirit and principle of the present invention shall fall within the protective scope of the present invention.

1. An application of a conjugate of PEG (polyethylene glycol) and rapamycin in preparation of a medicament for reducing immune response.

2. The application according to claim 1, characterized in that the conjugate of PEG and rapamycin has the structure shown in Formula I below:
PEG-X-D (I)
wherein,
PEG is a polyethylene glycol residue,
D is a residue of rapamycin or a derivative thereof,
X is a linking group between PEG and D.

3. The application according to claim 2, wherein in the conjugate, X has the following structure: —X1-X2-X3-, wherein, each of X1 and X3 is independently selected from one or a combination of several ones of —(CR1R2)a-, —(CH2)aNH—, —(CH2)aNHCO—, —(CH2)aCONH—, —(CH2)aCO—, —(CH2)aCOO— and —(CH2)aO, X2 is selected from cycloalkyl, aryl, heterocyclic, -A- and —Op-, each of R1 and R2 is independently selected from one or a combination of more than two of —H, C1-6 alkyl, —OR′—NHR′, —N(R′)2, —CN, —F, —Cl, —Br, —I, —COR′, —COOR′, —OCOR′, —CONHR′ and —CON(R′)2, R′ is selected from —H, C1-6 alkyl, —F, —Cl, —Br and —I,
a is an integer of 0-10,
-A- is an amino acid residue,
—Op- is an oligopeptide residue, with a structure below: 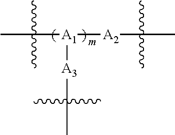
wherein, m is 1, 2 3 or 4,
A1 is selected from a residue of aspartic acid, glutamic acid and lysine,
each of A2 and A3 is independently same or different amino acid residue.

4. The application according to claim 3, wherein the conjugate has the following structure:

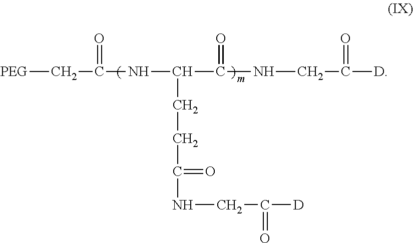

5. The application according to claim 1, wherein the immune response is an excessive immune response caused by foreign immunogen, and the foreign immunogen is selected from therapeutic agents, implants, artificial antigen carriers and toxins.

6. The application according to claim 5, wherein the therapeutic agent is a therapeutic protein, selected from one or more of cytokine, human hemoglobin, blood factor or blood coagulation factor, vascular endothelial growth factor antibody antagonist, hormone, antibody, enzyme and coenzyme.

7. The application according to claim 6, wherein the blood factor or blood coagulation factor is selected from one or more of factor I, factor II, tissue factor, factor V, factor VII, factor VIII, factor IX, factor X, factor Xa, factor XII, factor XIII, recombinant coagulation factor, von Willebrand factor, prekallikrein, high molecular weight kininogen, fibronectin, antithrombin III, albumin II, protein C, protein S, protein Z, protein Zs-related protease inhibitor (ZPI), plasminogen, α-2-antiplasmin, tissue plasminogen activator (tPA), urokinase, plasminogen activator inhibitor-1 (PAI1), plasminogen activator inhibitor-2 (PAI2), cancer procoagulant and epoetin Alfa.

8. The application according to claim 6, wherein the antibody comprises a monoclonal antibody, a polyclonal antibody, a dipolymer, a polymer, a bispecific antibody, a polyspecific antibody and an antibody fragment.

9. The application according to claim 6, wherein each of the enzyme and coenzyme is independently selected from one or more of imiglucerase, α-galactosidase A, agalsidase β, acid α-glucosidase, recombinant glucosinolate α, α-glucosidase, phenol sulfatase, aldurazyme, elaprase, galsulfase, PEGylated recombinant uricase, PEGylated recombinant Candida uricase and L-Asparaginasum.

10. The application according to claim 5, wherein the artificial antigen carrier is selected from keyhole limpet hemoeyanin, bovine serum albumin, human serum albumin and ovalbumin.

11. The application according to claim 10, wherein the conjugate has the artificial antigen carrier is keyhole limpet hemoeyanin.

12. An application of a conjugate of PEG (polyethylene glycol) and rapamycin in preparation of a medicament for strengthening the effect of a first medicament, wherein the first medicament is selected from at least one of cytokine, human hemoglobin, blood factor or blood coagulation factor, vascular endothelial growth factor antibody antagonist, hormone, antibody, enzyme and coenzyme.

13. The application according to claim 12, wherein the conjugate of PEG and rapamycin has the structure shown in Formula I below:
PEG-X-D (I)
wherein,
PEG is a polyethylene glycol residue,
D is a residue of rapamycin or a derivative thereof, and
X is a linking group between PEG and D.

14. The application according to claim 13, wherein the blood factor or blood coagulation factor is selected from one or more of factor I, factor II, tissue factor, factor V, factor VII, factor VIII, factor IX, factor X, factor Xa, factor XII, factor XIII, recombinant coagulation factor, von Willebrand factor, prekallikrein, high molecular weight kininogen, fibronectin, antithrombin III, albumin II, protein C, protein S, protein Z, protein Z-related protease inhibitor (ZPI), plasminogen, α-2-antiplasmin, tissue plasminogen activator (tPA), urokinase, plasminogen activator inhibitor-1 (PAI1), plasminogen activator inhibitor-2 (PAI2), cancer procoagulant and epoetin Alfa, preferably selected from factor VIII.

15. The application according to claim 12, wherein the antibody comprises a monoclonal antibody, a polyclonal antibody, a dipolymer, a polymer, a bispecific antibody, a polyspecific antibody and an antibody fragment; preferably, the monoclonal antibody is adalimumab.

16. The application according to claim 12, wherein each of the enzyme and coenzyme is independently selected from one or more of imiglucerase, α-galactosidase A, agalsidase β, acid α-glucosidase, recombinant glucosinolate α, α-glucosidase, phenol sulfatase, aldurazyme, elaprase, galsulfase, PEGylated recombinant uricase, PEGylated recombinant Candida uricase and L-Asparaginasum, and preferably selected from α-galactosidase A, acid α-glucosidase, EGylated recombinant uricase, PEGylated recombinant Candida uricase or L-Asparaginasum.

17. The application according to claim 12, wherein the first medicament is for treating tumors, hematologic diseases, infectious diseases, immune system diseases or metabolic diseases.

18. The application according to claim 17, wherein the tumors are selected from lung cancer, kidney cancer, melanoma, liver cancer, head and neck cancer, skin cancer, squamous-cell carcinoma, ovarian cancer, bone cancer, colorectal cancer, bladder cancer, stomach cancer, pancreatic cancer, prostate cancer, Hodgkin lymphoma, follicular lymphoma, chronic or acute leukemia, mesothelioma, pancreatic cancer, breast cancer, multiple myeloma and other tumors;
the hematologic disease is hemophilia;
the infectious diseases are selected from diseases caused by HIV infection, diseases caused by hepatitis virus infection, diseases caused by herpes virus infection and diseases caused by influenza virus infection;
the immune system diseases are selected from lupus erythematosus, rheumatoid arthritis, ankylosing spondylitis, asthenic bulbar paralysis, multiple sclerosis, autoimmune haemolytic anaemia, autoimmune hepatitis, scleroderma, polyarteritis nodose and Wegener granuloma;
the genetic diseases are selected from Fabry disease, Pompe disease, lysosomal storage diseases, genetic muscle diseases and genetic metabolic diseases; and,
the metabolic diseases are selected from disorders of amino acid metabolism, glycogen-storage disease I, malabsorption, oligosaccharidase deficiency, fructose metabolism disorders, galactose metabolism disorders, galactosemia, carbohydrate metabolism disorders, hypoglycemia, pyruvate metabolism disorders, hypolipidemia, hypolipoproteinemia, hyperlipemia, hyperlipoprotememia, carnitine or acylcarnitine translocase deficiency, porphyrin metabolism disorders, disorders of porphyrin and purine metabolism, lysosomal disease, nervous and nervous system diseases, sulfatide lipidosis, leukoencephalopathy and Lesh-nain syndrome.
